(19) United States
(12) Patent Application Publication
(10) Pub. No.: US 2010/0310621 A1
(43) Pub. Date: Dec. 9, 2010

(54) Controlled Iodine Release Particle Micro-Biocide

(76) Inventor: James C. Richards, Sudbury, MA (US)

Correspondence Address: CLIFFORD H. KRAFT, 320 ROBIN HILL DR., NAPERVILLE, IL, 60540, US

(21) Appl. No.: 12/794,528

(22) Filed: Jun. 4, 2010

Related U.S. Application Data
(60) Provisional application No. 61/184,286, filed on Jun. 4, 2009.

(51) Int. Cl. A61K 9/14; A61K 33/18; A61P 31/00

(52) U.S. Cl. 424/418; 424/668; 424/670; 424/671; 977/773; 977/906

(57) ABSTRACT

A controlled release molecular iodine biocide for use in human cavities especially the vaginal, penile canal, nasal passages and rectum. Particles can be functionalized with epithelial cell binding molecules and lymphocyte-binding molecules attached to their surfaces. Half of all the particles can contain pH buffer salts that control pH to between around pH 3.5 to pH 4.5. A second half of the particles can contain Iodide and iodate salts. When pH particles and iodate/iodide particles are bound next to each other, molecular iodine is released in a controlled fashion. Molecular iodine is effective biocide. In an alternate embodiment, there can be two distinct populations of particles, one with epithelial cell binding molecules and one with lymphocyte-binding molecules. Again, half of all the particles can contain a pH buffer and half iodide and iodate salts.

This application is related to and claims priority from U.S. Provisional Patent Application No. 61/184,286 filed Jun. 4, 2009. Application 61/184,286 is hereby incorporated by reference.

### BACKGROUND

1. Field of the Invention

The present invention relates generally to biocides and more particularly to a biocide that can be used in any mucous membrane/epithelial cell environment. The present invention is particularly directed to protect the female vaginal vault and cervix and the human rectum and penile canal as well as the nasal cavity from infection.

2. Description of the Prior Art

Biocides with applications to the female vagina, cervix and the human rectum and male penile canal are known in the art. Numerous compounds known to kill organisms are used in such applications. However, many of these compounds have difficulties associates with their use.

Targeted nanoparticles have been previously used to apply drugs. Farokhzad states the following in 2006 (Farokhzad et al. 2006, “Targeted nanoparticle-aptamer bioconjugates for cancer chemotherapy in vivo”, PNAS.):
“Targeted uptake of therapeutic nanoparticles in a cell-, tissue-, or disease-specific manner represents a potentially powerful technology. Using prostate cancer as a model, we report docetaxel (Dtxl)-encapsulated nanoparticles formulated with biocompatible and biodegradable poly(d,l-lactic-co-glycolic acid)-b/ock-poly(ethylene glycol) (PLGA-b-PEG) copolymer and surface functionalized with the A10 2′-fluoropyrimidine RNA aptamers that recognize the extracellular domain of the prostate-specific membrane antigen (PSMA), a well characterized antigen expressed on the surface of prostate cancer cells. These Dtxl-encapsulated nanoparticle-aptamer bioconjugates (Dtxl-NP-Apt) bind to the PSMA protein expressed on the surface of LNCaP prostate epithelial cells and get taken up by these cells resulting in significantly enhanced in vitro cellular toxicity as compared with nontargeted nanoparticles that lack the PSMA aptamer (Dtxl-NP) (P<0.0004)” Farokhzad et al. 2006

Molecular Iodine is known in the art as a biocide that is long-lasting with very few undesirable side effects. It would be advantageous to have a controlled iodine release polymer that uses delivery nanoparticles too large for endocytosis that are specifically designed to remain outside of cells. It would also be advantageous to use alternative epithelial cell binding substances that are attached to the particles via covalent linkage or biotin-streptavidin.

### SUMMARY OF THE INVENTION

The present invention relates to a controlled release molecular iodine biocide for use in human cavities especially the vaginal, penile canal, nasal passages and rectum. Particles, which can be polymer or other materials, can be functionalized with epithelial cell binding molecules and lymphocyte-binding molecules attached to their surfaces. Half of all the particles can contain pH buffer salts that control pH to between around pH 3.5 to pH 4.5. A second half of the particles can contain Iodide and iodate salts. When pH particles and iodate/iodide particles are bound next to each other the following chemical reaction occurs: IO₃⁻+5I⁻+6H⁺←→>>>3I₂+3H₂0 releasing molecular iodine in a controlled release fashion. Molecular iodine reacts very rapidly with multiple biochemical targets including all proteins and membranes thus acting as an effective biocide. In an alternate embodiment, there can be two distinct populations of particles, one with epithelial cell binding molecules and one with lymphocyte-binding molecules. Again, half of all the particles can contain a pH buffer and half iodide and iodate salts.

### DESCRIPTION OF THE FIGURES

Attention is now directed to several illustrations that aid in understanding the features of the present invention:

FIG. 1 shows an embodiment of the present invention using Epithelial cell specific aptamers.

FIG. 2 shows an embodiment of the present invention using Epithelial cell specific-binding molecules.

FIG. 3 shows an embodiment of the present invention using Lymphocyte cell specific-binding molecules.

Several drawings and illustrations have been presented present features of the invention. The scope of the present invention is not limited to what is shown in the figures.

### DESCRIPTION OF THE INVENTION

The present invention is distinctly different from Farokhzad et al., 2006, and a major improvement, in several important ways. First, all particles, which can be polymer or other material, are designed to be too large for cell endocytosis and in fact particles are specifically designed to remain outside targeted cells. Second, alternative epithelial cell binding substances are used such as actin, laminin, collagen and fibronectin and these are attached to the particles via covalent linkage or biotin-streptavidin. Particles are 50 to 100 μm are prepared containing a variety of chemicals. The 50 μm particles are preferred. Particles may be either biodegradable or non-biodegradeable but in all cases they carry chemicals both on their surface and throughout their structure. The described structures e.g., biodegradable poly(d,l-lactic-co-glycolic acid)-b/ock-poly(ethylene glycol) (PLGA-b-PEG) copolymer are a preferred embodiment but there are many structures that can support the chemicals described herein. Third, particles can have more than one binding ligand attached to the surface e.g., epithelial-specific ligands and lymphocyte-specific ligands. Fourth, more than one population of particles is always used to create the “iodine-generating factories”.

The present invention uses two distinct populations of particles. Particle population one particles are functionalized with epithelial cell binding molecules e.g., actin, fibronectin, etc. These particles have vaginal epithelial cell binding moieties specific for cell surface molecules such as epithelial glycoprotein. The epithelial cell membrane binding moieties are also found in the rectum and penile canal. The same population of particles is also functionalized with lymphocyte cell binding moieties such as glycosaminoglycan (GAG)—binding molecules including hyaluronic acid, chondroitin-4-sulfate, chondroitin-6-sulfate and heparin. All particles contain both populations of cell-specific binding molecules. For example, particles might have both actin and heparin equally dispersed on the particle surface. Alternatively, distinct particle populations can be employed each with only one cell-specific ligand attached to the particle surface. In this case particles would have only epithelial cell-specific binding moiety e.g., actin. A separate population of particles would have only lymphocyte-specific binding moiety e.g., heparin. Particle population one contains pH buffer salts that control pH at pH 3.5 to pH 4.5. For example, acetate buffer (acetic acid and sodium acetate) or citrate buffer (citric acid and sodium citrate) are preferred to maintain the pH around 4.0. Particle population two contains iodate and iodide salts (such as sodium or potassium iodide and iodate). The particles containing pH buffer salts have sufficient chemical to maintain pH at 4.0 for at least two weeks. Likewise, the particles containing iodide and iodate salts have sufficient chemicals to maintain I₂ creation for two weeks. When pH particles and iodate/iodide particles are bound next to each other the following chemical reaction occurs:

IO₃⁻+5I⁻+6H⁺←→>>>31₂+3H₂0 (1)

Molecular iodine reacts very rapidly with multiple biochemical targets including all proteins and membranes. These interactions include:
Reacts with SH-groups in proteins and prevents di-sulfide bridges (S—S) from forming and prevents protein function;
Attacks carbon-carbon double bonds (C═C) in unsaturated fatty acids disrupting membrane function and thus changes the physical properties of lipids and membrane immobilization;
Reacts very rapidly with the phenolic group of tyrosine forming monoiododerivitives and diiododeritives of tyrosine and such modified tyrocine-containing proteins experience steric hinderance with substrates or binding sites (Gottardi. 1991).

The present invention is a system and method for controlled release of molecular iodine to prevent microbial infection. A preferred use is to prevent microbial infections in the vagina and rectum of women and rectum and penile canal of men. The iodine described herein is molecular iodine also known as free iodine or I₂. For all discussions herein, molecular iodine will be referred to as free iodine. Also, free iodine is the predominate microbicide in any iodine composition such as polyvinyl pyrrolidone-iodine (PVP-iodine). Previous investigators have used 1% PVP-iodine to disinfect the vagina prior to childbirth to prevent HIV transmission form mother to child. Other investigators have used 1% PVP-iodine to disinfect the vagina prior to childbirth to reduce the risk of newborn child infection from pathogens residing inside the maternal vagina. The use of PVP-iodine reacts directly to kill pathogens. PVP-iodine is very effective in removing bacteria, viruses and fungi from inside the vagina and surface of the cervix. PVP-iodine does not persist inside the vagina and on the surface of the cervix. Consequently, once PVP-iodine is used to disinfect the vagina and cervix the disinfecting properties rapidly dissipate, and unless repeated applications of PVP-iodine are applied, the vagina and cervix become susceptible to new infections e.g., HIV, chlamydia, GC, syphilis, herpes, HPV, etc. Furthermore, not recognized before is that once a disinfecting agent like PVP-iodine is used, all the bacteria and viruses are killed inside the vagina including bacteria like Lactobacillus spp. Lactobacilli produce lactic acid, which maintains a low vaginal pH e.g., 4.0 and inhibits the growth of many microorganisms, including those that cause bacterial vaginosis. In addition, some lactobacilli also produce hydrogen peroxide, which is toxic to a number of microorganisms including HIV. The use of agents like PVP-iodine and 0.2% chlorhexidine to disinfect the vagina effectively disinfect the vaginal vault but also remove all the resident lactobacilli and this results in an elevated vaginal vault pH and opportunistic pathogens enter and grow. Examples include Candida albicans and pathogens associated with bacterial vaginosis such as Gardnerella vaginalis and Microplasma hominis.

The present invention kills all pathogens inside the vaginal vault and on surface of the cervix but also will maintain the pH at pH 3.5-4.5 over time and thus maintain the low pH environment that resists re-colonization by harmful microorganisms. Furthermore, since low levels of free iodine e.g., 2-4 μg/mL are created from iodate and iodide containing particles. As stated earlier, free iodine synthesis is favored at pH 4.0. Therefore, the present invention creates the following microbial ecosystem:
A mixture of particles containing chemicals is inserted into the vagina. One population of particles contain pH buffering salts and a second population of particles contain iodate and iodide salts.
The particles are randomly distributed inside the vaginal vault and eventually rest on and bind to the mucosal surface composed of epithelial cells. Some particles attach to epithelial cells via epithelial cell binding molecules. These epithelial cell binding molecules are attached to the particles and are designed to bind to epithelial cell surface binding sites.
Particles pH are in greater abundance than particles with iodate and iodide. In general we find that a ratio of 3:1 is effective. The result is that membrane-bound particles iodate and iodide are always adjacent to membrane-bound particles pH and this configuration promotes free iodine synthesis.
The vaginal/cervix environment now gently reaches s stable pH of 3.5-4.0. At this pH free iodine is synthesized and kills any pathogens present on contact and provides a surveillance level of free iodine protection if any new pathogens enter the vaginal vault.
The present invention contemplates that the pH and free iodine generation will continue to be generated over time. We envision particles that can continue to generate pH and free iodine for two weeks.
The pH buffering capacity of the present invention is sufficient to overcome the pH of human semen that has a pH of pH>7 and maintain the pH at pH 4 and this is important to protect against HIV and STDs.
If the present invention is used on an ongoing basis e.g., weekly then lactobacilli may no longer be necessary as symbiotic bacteria to maintain vagina pH. However, a companion application of this invention would be a composition containing only particles pH that would maintain the pH and promote recolonization by lactobacilli. This companion application would not contain particles iodate and iodide.

The present invention thus relates to a mixture of particles that have both epithelial cell-binding molecules and lymphocyte-binding molecules attached to the particle surface. Half of these particles have pH buffer salts incorporated into their structure and the other half has iodide and iodate salts incorporated into their structure. In this composition of matter pH particles and iodine particles will be distributed throughout the vaginal vault and bind to epithelial cells lining the vagina. Iodine and pH particles will be bound next to each other 66% of the time and thus will become “Iodine Factories” and generate free iodine over time thus providing a constant level of antiseptic iodine over time up to two weeks. Furthermore, if lymphocytes are inoculated into the vagina by semen during unprotected vaginal intercourse the lymphocytes would become bound to particles attached to the vaginal wall. If the lymphocytes are infected with HIV then budding HIV virions will be immediately killed by iodine as they egress from inside the lymphocyte.

An alternative embodiment of the invention contains distinct populations of particles. One population has only epithelial cell-specific binding molecules e.g., actin and half have pH buffer salts incorporated into their structure and the other half have iodide and iodate salts incorporated into their structure. The second population of particles has only lymphocyte cell-specific binding molecules e.g., heparin and half have pH buffer salts incorporated into their structure and the other half have iodide and iodate salts incorporated into their structure.

A further embodiment of the present invention can be thought of as a “first aid” composition. If a female has unwanted vaginal intercourse where semen is inserted into the vagina, the present invention will immediately kill all pathogens in the vaginal vault. This is a two-part chemistry that requires a unique delivery device. Briefly, the two-chambered device that resembles a syringe with two chambers in the barrel of the syringe. A breakable membrane separates the chambers and each chamber contains a unique solution. Solution one contains a pH buffered solution containing glycerol and solution two contains free iodine in 100% ethanol. The delivery device is inserted into the vagina and when the plunger is depressed the solutions are mixed and dispensed into the vaginal vault. The resulting solution contains free iodine at 100 μg/mL and this level of free iodine will kill all pathogens and will “tan” or coat all free CD4 cells in the vagina and inhibit egress of HIV. This embodiment is stable at room temperature and can be kept in a purse or anywhere and if an unplanned vaginal event occurs then the woman can protect herself from any microbial infection.

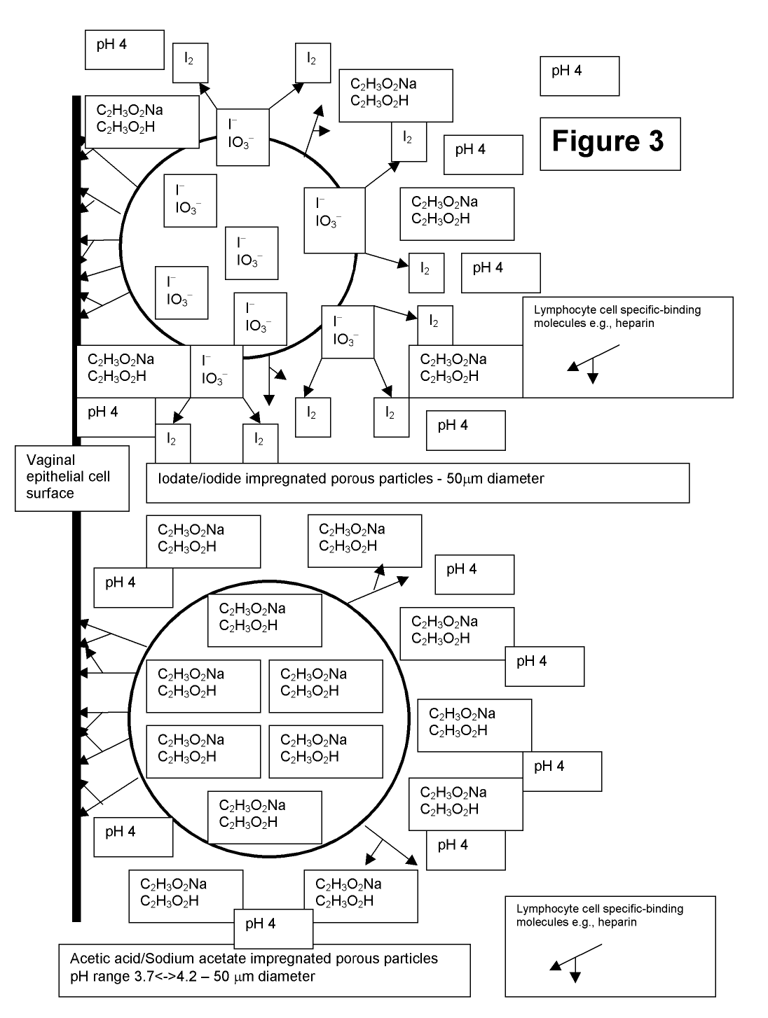

Most, if not all, previous attempts to prevent HIV infection of host cells have directed the action on preventing the HIV virion from infecting the host cell. The present invention is distinctly different from all previous attempts to prevent HIV infection of host cells. The present invention uses a combination of pH particles and iodine microbicide particles that bind to the surface of HIV-infected lymphocytes and create an antimicrobial environment that kills all HIV virions as they emerge from host lymphocytes. This is shown in FIG. 3.

The binding to lymphocytes is accomplished using lymphocyte-specific binding-molecules such as heparin. Particles containing both epithelial binding molecules and lymphocyte binding molecules are created to have both binding ligands on the same particles or can be on separate particles. The important distinction from all previous inventions is that the present invention kills HIV virions as they emerge from infected cells before they can infect new cells such as cells in the vagina or anus. In this regard, the present invention free iodine kills HIV before the virus can infect any new cell.

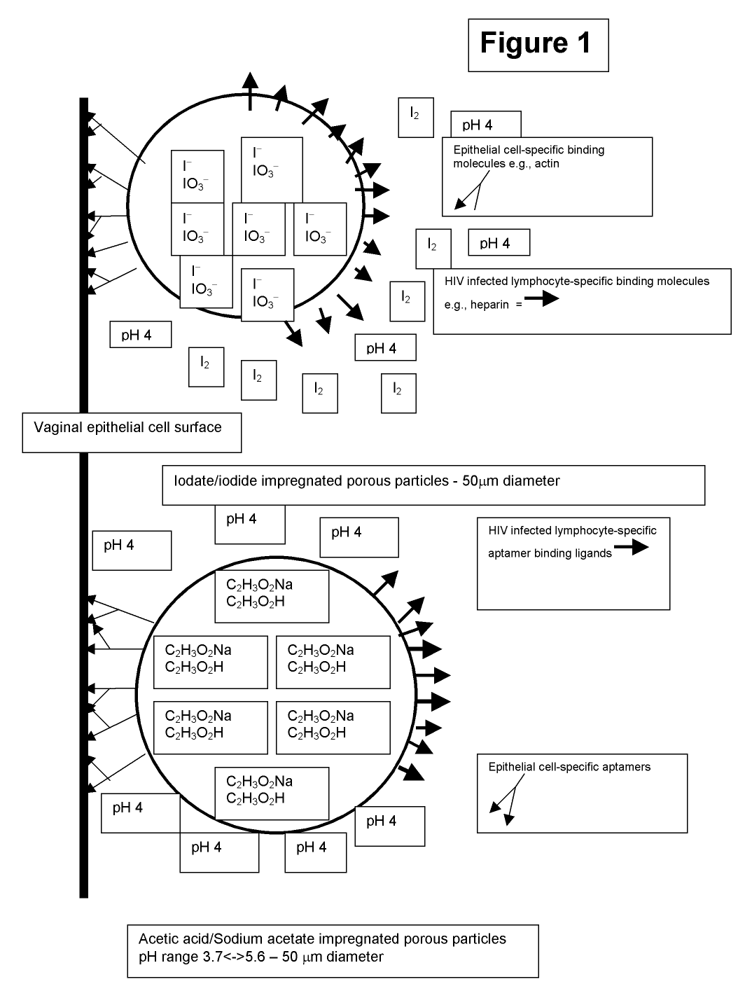

A further embodiment of the invention contains a mixture of particles that have both epithelial cell-binding molecules and lymphocyte-binding molecules attached to the particle surface. Half of these particles have pH buffer salts incorporated into their structure, and the other half has iodide and iodate salts incorporated into their structure. This is illustrated in FIG. 1.

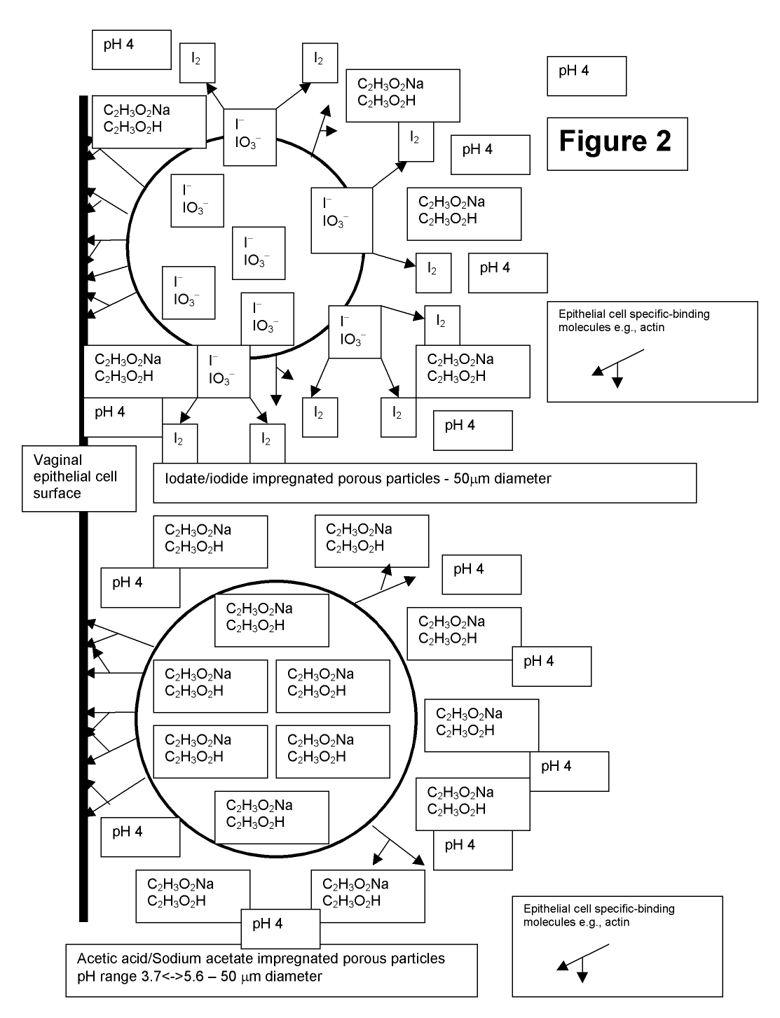

Another embodiment contains distinct populations of particles. One population has only epithelial cell-specific binding molecules e.g., actin and half have pH buffer salts incorporated into their structure, and the other half have iodide and iodate salts incorporated into their structure. This is illustrated in FIG. 2. The second population of particles has only lymphocyte cell-specific binding molecules e.g., heparin and half have pH buffer salts incorporated into their structure and the other half have iodide and iodate salts incorporated into their structure. This is illustrated in FIG. 3. Some of the features of the present invention are:
1. Two particle iodine—generation factory that is pH dependent. Creates anti-microbial iodine that is created over time.
2. Particles are not taken up by host cells—no endocytosis.
3. HIV-infected lymphocytes bind to Iodine Factory particles and HIV is killed as it buds from inside the lymphocyte.
   4. Molecular iodine kills all microbial pathogens—HIV-bacteria-all STDs
5. Time-release I₂ creates a surveillance system.
6. pH 4.0 maintained in the vaginal vault to promote I₂ surveillance.
7. The I₂ is not cytotoxic and non-irritating.

Several descriptions and illustrations have been presented to aid in understanding the present invention. One with skill in the art will realize that numerous changes and variations are possible without departing from the spirit of the invention. Each of these changes and variations is within the scope of the present invention.

I claim:

1. A controlled-release molecular iodine system comprising:
a mixture of nanoparticles particles that have both epithelial cell-binding molecules and lymphocyte-binding molecules attached to their surfaces; wherein,
half of these particles contain a pH buffer, and half contain iodide and iodate salts.

2. The system of claim 1 wherein said half containing a pH buffer contains a pH buffer that controls pH to between around pH 3.5 to around pH 4.5.

3. The system of claim 2 wherein said pH buffer contains acetic acid and sodium acetate or citric acid and sodium citrate.

4. The system of claim 1 wherein said particles are biodegradable.

5. The system of claim 1 wherein said epithelial cell binding molecules are actin or fibronectin.

6. The system of claim 1 wherein said lymphocyte cell binding molecules include hyaluronic acid, chondroitin-4-sulfate, chondroitin-6-sulfate or heparin.

7. The system of claim 1 wherein said iodide salt is potassium or sodium iodide, and said iodate salt is potassium or sodium iodate.

8. The system of claim 1 wherein said nanoparticles have diameters of around 50-100 μm.

9. A controlled-release molecular iodine system comprising:
a mixture of two populations of nanoparticles particles where particles of a first population has epithelial cell-binding molecules attached to their surfaces and particles of a second population has lymphocyte-binding molecules attached to their surfaces; wherein,
half of all of these particles contain a pH buffer, and half contain iodide and iodate salts.

10. The system of claim 9 wherein said half containing a pH buffer contains a pH buffer that controls pH to between around pH 3.5 to around pH 4.5.

11. The system of claim 10 wherein said pH buffer contains acetic acid and sodium acetate or citric acid and sodium citrate.

12. The system of claim 9 wherein said particles are biodegradable.

13. The system of claim 9 wherein said epithelial cell binding molecules are actin or fibronectin.

14. The system of claim 9 wherein said lymphocyte cell binding molecules include hyaluronic acid, chondroitin-4-sulfate, chondroitin-6-sulfate or heparin.

15. The system of claim 9 wherein said iodide salt is potassium or sodium iodide, and said iodate salt is potassium or sodium iodate.

16. The system of claim 9 wherein said nanoparticles have diameters of around 50-100 μm.

17. A method of controlled release molecular iodine for body cavities comprising:
attaching molecules onto surfaces of a population of particles including epithelial cell-binding molecules and lymphocyte-binding molecules;
causing half said particles to contain a pH buffer and half said particles to contain iodide and iodate salts.

18. The method of claim 17 wherein said particles are between around 50 to 100 μm.

19. The method of claim 17 wherein said buffer controls pH to between around pH 3.5 to around pH 4.5.
